(19) United States
(12) Patent Application Publication
(10) Pub. No.: US 2024/0065429 A1
(43) Pub. Date: Feb. 29, 2024

(54) INTELLIGENT VISUALIZING ELECTRIC TOOTH BRUSH

(71) Applicant: Dan XIONG, Johns Creek, GA (US)

(72) Inventor: Dan XIONG, Johns Creek, GA (US)

(21) Appl. No.: 18/497,714

(22) Filed: Oct. 30, 2023

Related U.S. Application Data
(63) Continuation-in-part of application No. PCT/CN2021/114479, filed on Aug. 25, 2021.
(60) Provisional application No. 63/381,501, filed on Oct. 28, 2022.

(30) Foreign Application Priority Data
Jun. 17, 2021 (CN) 202110669700.X

(51) Int. Cl. A46B 15/00; G16H 15/00; G16H 30/40; G16H 50/20

(52) U.S. Cl. CPC A46B 15/0006; A46B 15/0008; A46B 15/004; G16H 15/00; G16H 30/40; G16H 50/20; A46B 2200/1066

(57) ABSTRACT

An oral health management system based on artificial intelligence image recognition to adjust the electric toothbrush includes an intelligent electric toothbrush and a server. The intelligent electric toothbrush has an image acquisition unit, an image judgment unit, a first communication unit, a positioning unit, a main control unit, a motor drive unit and a voice playback unit. The server has a recognition unit, a second communication unit and a parameter determination unit. The parameter determination unit is set on the intelligent electric toothbrush or server to determine the corresponding teeth cleaning parameters for different oral areas based on the recognition results. The system obtains a user's oral image and performs recognition and analysis, and then selects tooth cleaning parameters based on the recognition and analysis results to provide targeted oral cleaning services for the user.

### CROSS REFERENCE TO RELATED APPLICATIONS

This application is a continuation-in-part of PCT international application No. PCT/CN2021/114479, filed Aug. 25, 2021, which claims the benefit to the priority of Chinese patent application No. CN 202110669700.X, filed Jun. 17, 2021; and claims the benefit of U.S. provisional application No. 63/381,501, filed Oct. 28, 2022; the content of each is incorporated herein by reference in its entirety.

### TECHNICAL FIELD

The present disclosure relates to the technical field of electric toothbrushes, in particular to an oral health management system based on artificial intelligence image recognition to adjust the electric toothbrush.

### Background Art

With the increasing quality of life and people's demands for oral health protection, electric toothbrushes with good cleaning effects have emerged.

Currently, the electric toothbrushes on the market are unable to collect and analyze the oral health status of the user, nor can they provide targeted oral cleaning services based on the oral health status, and cannot achieve better oral cleaning effects. Moreover, they can only be used as cleaning tools and cannot provide prevention and motivation effects for users.

### SUMMARY

To address the above problems, the present disclosure proposes an oral health management system based on artificial intelligence image recognition to adjust the electric toothbrush.

In order to address at least one of the above technical problems, the disclosure provides the following technical proposal:

On one hand, the disclosure provides an oral health management system based on artificial intelligence image recognition to adjust the electric toothbrush, which comprises an intelligent electric toothbrush and a server;

Wherein, the intelligent electric toothbrush comprises the following:

an image acquisition unit for capturing oral images;

an image judgment unit for determining whether the captured oral image is a valid oral image. If yes, the image is sent by the first communication unit to the server for recognition. If not, the reason for the invalid image is sent to the main control unit.

a first communication unit used to the send valid oral image to the server and receive recognition results or dental cleaning parameters sent back by the server. The recognition result at least includes the information on the position of the teeth, information on the presence of caries and/or dental calculus, and information on the severity grading of caries and/or dental calculus. The teeth cleaning parameters include brushing duration and/or vibration frequency;

a positioning unit used to obtain the position information of the teeth being cleaned and send the information to the main control unit;

a main control unit used to select corresponding teeth cleaning parameters based on the position information of the teeth being cleaned and convert them into control signals to be sent to the motor drive unit in real time. In addition, upon receiving the reason for the invalid image sent by the image judgment unit, the main control unit (105) selects the corresponding voice data in the voice database based on the reason for the invalid image;

a motor drive unit (106) used to connect the motor and drive the motor to vibrate based on the control signal of the main control unit;

and a voice playback unit used for voice playback based on the voice data selected by the main control unit.

The server comprises the following:

a recognition unit for recognizing the received oral image and generating a recognition result. The recognition method includes the following steps: obtaining an oral image, determining the position information of caries and/or dental calculus through object detection algorithms, determining the severity grading information of caries and/or dental calculus through convolutional neural networks, and generating a recognition result;

a second communication unit for receiving the oral image sent by the intelligent electric toothbrush and sending a recognition result or tooth cleaning parameter to the intelligent electric toothbrush;

and a parameter determination unit for determining tooth cleaning parameters corresponding to different oral areas based on the recognition result. The parameter determination unit is set on an intelligent electric toothbrush or server.

Also, the disclosure provides a control method based on the artificial intelligence image recognition to adjust the intelligent electric toothbrush, and an oral health management system based on the artificial intelligence image recognition to adjust the intelligent electric toothbrush, which includes the following steps:

the intelligent electric toothbrush captures oral images;

the intelligent electric toothbrush judges whether the captured oral image is valid. If yes, the valid oral image is uploaded to the server. If not, the corresponding voice prompt is issued.

the server recognizes the received oral image and generates the recognition result. The recognition result at least includes the position information of the teeth, information on the presence of caries and/or dental calculus, and information on the severity grading of caries and/or dental calculus.

the server determines the corresponding teeth cleaning parameters for different oral areas based on the recognition result and sends them to the intelligent electric toothbrush, or the server sends the recognition result to the intelligent electric toothbrush. The intelligent electric toothbrush determines tooth cleaning parameters corresponding to different oral areas, including brushing duration and/or vibration frequency, based on the recognition result.

the intelligent electric toothbrush obtains the position information of the teeth currently being cleaned.

the intelligent electric toothbrush selects the corresponding teeth cleaning parameters based on the position information of the teeth currently being cleaned.

and the intelligent electric toothbrush controls motor vibration based on current teeth cleaning parameters.

Wherein, the server recognizes the received oral image and generates a recognition result. The process includes the following steps:

obtain an oral image;

determine the position information of caries and/or dental calculus through object detection algorithms;

determine the severity grading information of caries and/or dental calculus through convolutional neural networks;

and generate a recognition result.

At last, the disclosure provides an intelligent electric toothbrush, which can be applied to any oral health management system based on artificial intelligence image recognition to adjust the electric toothbrush, comprising the following:

an image acquisition unit for capturing oral images;

an image judgment unit for determining whether the captured oral image is a valid oral image.

If yes, the image is sent by the first communication unit to the server for recognition. If not, the reason for the invalid image is sent to the main control unit.

a first communication unit used to the send valid oral image to the server and receive recognition results or dental cleaning parameters sent back by the server. The recognition result at least includes the information on the position of the teeth, information on the presence of caries and/or dental calculus, and information on the severity grading of caries and/or dental calculus. The teeth cleaning parameters include brushing duration and/or vibration frequency;

a positioning unit used to obtain the position information of the teeth being cleaned and send the information to the main control unit;

a main control unit used to receive the reason for the invalid image, select the corresponding voice data in the voice database based on the reason for the invalid image, and select the corresponding teeth cleaning parameters based on the position information of the teeth being cleaned, convert them into control signals, and send them to the motor drive unit in real-time;

a motor drive unit (106) used to connect the motor and drive the motor to vibrate based on the control signal of the main control unit;

and a voice playback unit used for voice playback based on the voice data selected by the main control unit.

and a screen interactive system which displays the user's brushing times by small red flowers and small stars on the touch screen, making the children like brushing and have the brushing habit.

The advantages of the disclosure are as follows: the accurate oral health information related to dental caries, dental calculus, etc. of the user is obtained by obtaining the user's oral image, and the recognition and analysis of the oral image is obtained through object detection algorithms and convolutional neural networks. The intelligent electric toothbrush is controlled accordingly, providing targeted oral cleaning services for the user, improving the cleaning effect of the intelligent electric toothbrush, and improving the user experience. The advance judgment of the validity of the oral image by the image judgment unit avoids adverse effects on recognition results caused by unclear oral images, overexposure or darkness of images, and imaging angle issues, thereby improving the accuracy of recognition results. Through the analysis of invalid images by the image judgment unit, the voice playback unit is controlled to play related voice data, providing prompts to the user in the form of voice, facilitating the user to obtain more intuitive guidance and obtaining effective oral images. On the one hand, it further improves the accuracy of the recognition result, and on the other hand, it improves the user experience.

In addition, if not otherwise specified, the disclosed technical proposal can be realized by using conventional means in the art.

### DESCRIPTION OF DRAWINGS

To more clearly explain the technical proposal of the embodiments of the present disclosure, the drawings required for the description of the embodiments are briefly introduced. It is obvious that the drawings below are only for some embodiments of the present disclosure. The ordinary technicians in the field can also obtain other drawings from these drawings without any creative labor.

FIG. 1 is a schematic diagram of the structure of the oral health management system based on artificial intelligence image recognition to adjust the electric toothbrush provided in an embodiment of the disclosure.

FIG. 2 is a flowchart of the control method of the intelligent electric toothbrush provided by another embodiment of the disclosure.

FIG. 3 is a flowchart of step S13 of the control method for the intelligent electric toothbrush provided in an embodiment of the disclosure.

FIG. 4 is a flowchart of step S132 of the control method of the intelligent electric toothbrush provided in an embodiment of the disclosure.

FIG. 5 is a flowchart of step S133 in the control method of the intelligent electric toothbrush provided in an embodiment of the disclosure.

FIG. 6 is a schematic diagram of the structure of the intelligent electric toothbrush provided by another embodiment of the disclosure.

FIG. 7 is a functional schematic diagram of the electric toothbrush of the disclosure.

FIG. 8 is a flowchart of the image acquisition unit 101 of the disclosure.

FIG. 9 is a flowchart of the image judgment unit 102 of the disclosure.

FIG. 10 is a flowchart of the first communication unit 103 of the disclosure.

FIG. 11 is a flowchart of the positioning unit 104 of the disclosure.

FIG. 12 is a flowchart of the main control unit 105 of the disclosure.

FIG. 13 is a flowchart of the motor drive unit 106 of the disclosure.

FIG. 14 is a flowchart of the voice playback unit 107 of the disclosure.

FIG. 15 is a schematic diagram of the intelligent interactive system of the disclosure.

FIG. 16 is a flowchart of the recognition unit 201 of the disclosure.

FIG. 17 is a flowchart of the second communication unit 202 of the disclosure.

FIG. 18 is a flowchart of the parameter determination unit 108 of the disclosure.

### DETAILED DESCRIPTION OF EMBODIMENTS

In order to make the purpose, technical proposal and advantages of the disclosure clearer, the following is a further detailed explanation of the disclosure in conjunction with the accompanying drawings and embodiments. It should be understood that the specific embodiments described here are only part of the embodiments of the disclosure but not all of them, and are only used to explain the disclosure but not to limit it. Based on the embodiments in the disclosure, all other embodiments obtained by ordinary technical personnel in the art without creative labor should fall within the scope of protection of the disclosure.

It should be noted that the terms “include” and “have”, as well as any variations thereof, are intended to cover non-exclusive inclusion, for example, the process, method, device or server that includes a series of steps or units does not need to be limited to those clearly listed steps or units, but may include other steps or units that are not clearly listed or inherent to the process, method, product or device.

### Embodiment 1

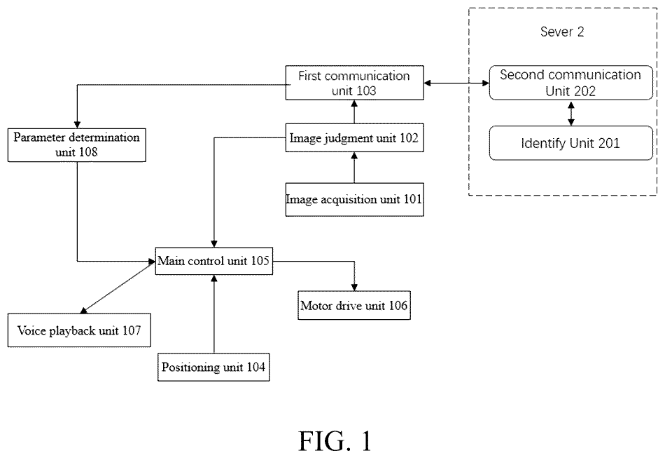

FIG. 1 shows the oral health management system based on artificial intelligence image recognition to adjust the electric toothbrush provided in the embodiment of the application, comprising an intelligent electric toothbrush 1 and a server 2.

Wherein, the intelligent electric toothbrush 1 comprises the following:

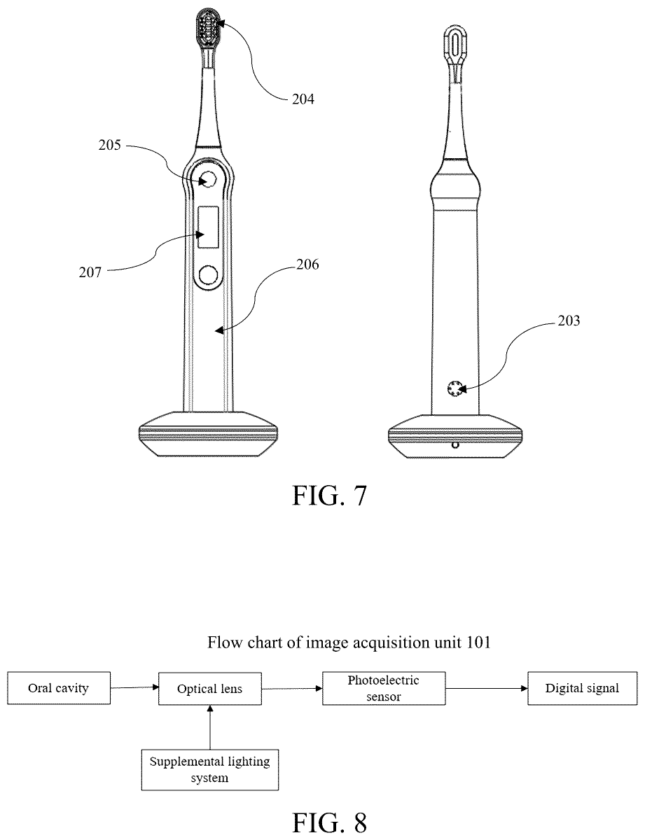

an image acquisition unit 101. Implemented on the product, the image acquisition camera 205 in FIG. 7 is used to capture the user's oral images. The optical sensor converts the optical image into a digital signal, which includes multi-dimensional features such as shape, color, position, brightness, etc.;

an image judgment unit 102 for determining whether the captured oral image is a valid oral image. If yes, the image is sent by the first communication unit 103 to the server 2 for recognition. If not, the reason for the invalid image is sent to the main control unit 105. Implemented on the product, that is, the PCBA integrated control circuit inside the body 206 in FIG. 7 identifies the digital model transmitted by 201. If it meets the requirements, it is uploaded to the server, and if it is invalid, it is returned to the main control unit. Finally, it is fed back to the consumer through the loudspeaker 203 at the back of the body.

a first communication unit 103 used to the send valid oral image to the server 2 and receive recognition results sent back by the server 2. The recognition result includes the information on the position of the teeth, information on the presence of caries and/or dental calculus, and information on the severity grading of caries and dental calculus.

a positioning unit 104 used to obtain the position information of the teeth being cleaned and send the information to the main control unit 105;

a main control unit 105 used to select corresponding teeth cleaning parameters based on the position information of the teeth being cleaned and convert them into control signals to be sent to the motor drive unit 106 in real time. In addition, upon receiving the reason for the invalid image sent by the image judgment unit 102, the main control unit 105 selects the corresponding voice data in the voice database based on the reason for the invalid image, and the user is reminded by a loudspeaker 203;

a motor drive unit 106 used to connect the motor and drive the motor to vibrate based on the control signal of the main control unit 105;

and a voice playback unit 107 used for voice playback based on the voice data selected by the main control unit 105; and

a parameter determination unit 108 for determining the corresponding teeth cleaning parameters for different oral areas based on the the recognition result sent back by the server 2. Tooth cleaning parameters include brushing duration and vibration frequency;

The server comprises the following:

a recognition unit 201 for recognizing the received oral image and generating a recognition result. The recognition method includes the following steps: obtaining an oral image, determining the position information of caries and dental calculus through object detection algorithms, determining the severity grading information of caries and dental calculus through convolutional neural networks, and generating a recognition result;

and a second communication unit 202 for receiving the valid oral image sent by the intelligent electric toothbrush and sending a recognition result to the intelligent electric toothbrush;

The advantages of the disclosure are as follows: the accurate oral health information related to dental caries, dental calculus, etc. of the user is obtained by obtaining the user's oral image, and the recognition and analysis of the oral image is obtained through object detection algorithms and convolutional neural networks. The intelligent electric toothbrush is controlled accordingly, providing targeted oral cleaning services for the user, improving the cleaning effect of the intelligent electric toothbrush, and improving the user experience. The advance judgment of the validity of the oral image by the image judgment unit avoids adverse effects on recognition results caused by unclear oral images, overexposure or darkness of images, and imaging angle issues, thereby improving the accuracy of recognition results. Through the analysis of invalid images by the image judgment unit, the voice playback unit is controlled to play related voice data, providing prompts to the user in the form of voice, facilitating the user to obtain more intuitive guidance and obtaining effective oral images. On the one hand, it further improves the accuracy of the recognition result, and on the other hand, it improves the user experience.

In optional embodiments, the image acquisition unit 101 may be a miniature wide-angle camera 205, installed on the body of the intelligent electric toothbrush. As a result, it can obtain relatively complete oral images without the need for professional dental imaging equipment.

In optional embodiments, the lens of the image acquisition unit 101 can be made of crystal glass with an additional evaporated antireflective film. The crystal glass has excellent anti-fog performance and the antireflective film technology, which avoids the unclear oral images obtained due to the presence of water mist in the human mouth, further improving the accuracy of recognition results.

In optional embodiments, a light replenishing module is installed around the image acquisition unit 101 to ensure sufficient light during shooting, thereby improving the clarity of the oral image, avoiding excessive darkness of the oral image, ensuring the acquisition effect of the oral image, and further improving the accuracy of the recognition result.

In optional embodiments, the image judgment unit 102 can determine whether the oral image is valid, including determining whether the oral image is clear, determining whether the oral image is overexposed or overdark, and determining whether the imaging angle of the oral image is appropriate. Specifically, presetting oral image features includes but not limited to clarity, exposure and imaging angle, as well as related parameter thresholds for shape and position features. Based on the preset parameter thresholds, the various conditions for the validity of the oral image are sequentially judged. If the oral image meets the requirements of clarity, moderate exposure and imaging angle, the oral image is judged to be valid and sent to the server for recognition. If the oral image does not meet at least one of the above three judgment conditions, it is determined that the oral image is invalid, and the image judgment unit 102 sends the reason for the invalidity to the main control unit 105. When there is more than one reason for the invalid oral image, the image judgment unit 102 sends all the invalid reasons of the oral image to the main control unit 105.

In optional embodiments, the image acquisition unit 101 obtains images in JPG format. In the image judgment unit 102, whether the oral image is clear can be determined by whether the pixels of the oral image meet 720*720. Whether the oral image is overexposed or overdark can be determined by whether the brightness of the oral image meets 200cd/square meter. The suitability of the imaging angle of oral images can be determined by whether the imaging angle size is within the range of greater than 50° and less than or equal to 90°, with the reference of the imaging angle being the occlusal surface of the teeth. Therefore, setting the related specifications and parameters of the oral images can judge the validity of the oral image by the intelligent electric toothbrush, providing a basis for accurate and effective recognition of oral images in the future.

In optional embodiments, when the reason for invalidity is the unclear oral image, the main control unit 105 selects the corresponding voice data in the voice database, which can include adjustment prompts for shooting duration or refocusing. When the reason for invalidity is overexposure or excessive darkness of the oral image, the main control unit 105 selects the corresponding voice data in the voice database, which can include adjustment prompts for the user's mouth opening size and adjustment prompts for turning on or off the light replenishing module. When the reason for invalidity is that the imaging angle of the oral image is not appropriate, the main control unit 105 selects the corresponding voice data in the voice database, which can include adjustment prompts for the user's photo posture, such as “Please extend the toothbrush head inward”, etc. Therefore, the reason for the invalid oral image corresponds to the voice data for adjustment prompt. Targeted prompts are provided to the user to assist in shooting valid oral images. On the one hand, the quality of oral images is improved, further improving recognition accuracy. On the other hand, voice prompts are provided to the user, making it more direct and effective, and improving user experience.

In optional embodiments, the main control unit 105 can directly control the light replenishing module to turn on or off or directly control the image acquisition unit 101 to adjust the focus based on

the type of invalid reason for the oral image after receiving the invalid reason for the image sent by the image judgment unit 102. As a result, it reduces the difficulty of user operation, makes it more convenient to use, and improves the user experience.

In optional embodiments, when the main control unit 105 directly controls the light replenishing module to open or close based on the type of invalid oral image or directly controls the image acquisition unit 101 to adjust focus, the corresponding waiting prompt voice can be selected in the voice database, such as “In automatic focusing, please maintain this posture”, etc. After the opening and closing actions of the light replenishing module or the focusing of the image acquisition unit 101 is completed, the corresponding shooting prompt voice can be selected in the voice database, such as “Refocused, please take a photo”, etc. The specific method of replenishing light is to automatically determine the level of current of the replenishing light and control its brightness by identifying the lighting conditions. The focal length of the camera is automatically adjusted by a micro motor.

In optional embodiments, the positioning unit 104 may include a gyroscope for obtaining the position, movement trajectory and acceleration parameters of the intelligent electric toothbrush. Thus, the position information of the teeth being cleaned is determined based on the posture parameters during the use of the intelligent electric toothbrush, and the corresponding vibration mode is selected based on the recognition result of caries or dental calculus in the teeth at that position sent back by server 2. The motor drive unit is controlled to drive the motor to vibrate at the corresponding frequency of this mode. It provides corresponding oral cleaning services for different tooth conditions through the brush head 204 at the end, improving the user experience.

In optional embodiments, the intelligent electric toothbrush 1 further comprises a button operation module 109 for receiving the user's button operation signal and sending it to the main control unit. In addition, the main control unit 105 can receive the button operation signal and convert it into a corresponding control signal to be sent to the image acquisition unit, motor drive unit and voice playback unit.

In optional embodiments, the intelligent electric toothbrush 1 may comprise a first button and a second button. The button operation module 109 receives user operations on the first and second buttons and sends them to the main control unit 105 which controls the image acquisition unit 101, motor drive unit 106 and voice playback unit 107 to operate.

Specifically, when the power off state is set, briefly press the first button to enter the shooting mode. At this time, the voice playback unit 107 plays the corresponding voice prompt “Hello, please take a photo”; short press the second button, and at this time, the voice playback unit 107 plays the corresponding photo taking sound, and the image acquisition unit 101 performs oral image acquisition. If the image judgment unit 102 determines that the image is invalid, the voice playback unit 107 plays the related voice prompt “Please adjust the posture of the electric toothbrush and take photos”. If the image judgment unit 102 determines that the image is valid, the image is uploaded by the first communication unit 103. After the upload is successful, the “Upload successful” prompt sound appears. If the image is not successfully uploaded for more than 5 seconds, the “Upload failed” prompt sound appears.

In the shooting mode, briefly press the first button to enter the brushing mode. At this time, the motor drive unit 106 controls the motor to start vibrating. The motor can be set to pause once every predetermined time to remind the user to switch the parts to be cleaned. In the brushing mode, short press the second button, and the main control unit 5 controls the motor to change the vibration mode by controlling the control instruction sent to the motor drive unit 106.

In optional embodiments, the vibration mode of the motor can be divided into the first vibration mode, the second vibration mode, and the third vibration mode, corresponding to three vibration frequencies. Specifically, it can be set in the way that the vibration frequency of the first vibration mode is greater than the vibration frequency of the second vibration mode, and the vibration frequency of the second vibration mode is greater than the vibration frequency of the third vibration mode.

In optional embodiments, the intelligent electric toothbrush 1 also comprises a timing module, which is connected to the main control unit 105. The main control unit 105 sets the timing module based on the brushing time in the teeth cleaning parameters. When the time calculated by the timing module reaches the preset brushing time, the main control unit 105 automatically controls the brushing time by controlling the motor drive unit 106 to stop the vibration of the motor.

In optional embodiments, the intelligent electric toothbrush 1 also comprises a default frequency setting module for receiving and memorizing the adjustment instructions for the vibration frequency or vibration mode. When the adjustment instruction for the same vibration frequency or vibration mode occurs multiple times, the vibration frequency or its corresponding vibration mode is set as the default vibration frequency or default vibration mode. The adjustment instruction can be issued by the user by operating the first or second button. Therefore, it can automatically adjust the vibration frequency or mode according to user habits, improving the user experience.

In optional embodiments, the voice content played by the voice playback unit 107 may also include one or more of the oral health warm prompts and brushing guidelines.

In optional embodiments, the parameter determination unit 108 can be set on the server to determine the corresponding teeth cleaning parameters for different oral areas based on the recognition result. At this time, the second communication unit 202 sends the teeth cleaning parameters, and correspondingly, the first communication unit 103 receives the teeth cleaning parameters sent back by the server. Therefore, setting the parameter determination unit 108 on the server can make the server not limited by the specifications of the intelligent electric toothbrush, thus making the calculation speed faster and reducing the data storage pressure of the intelligent electric toothbrush.

In optional embodiments, the recognition result generated by the recognition unit 201 can also only include the information on the position of the teeth, whether there is caries in the teeth, and the severity grading information of the caries, or only include the information on the position of the teeth, whether there is dental calculus in the teeth, and the severity grading information of the dental calculus. Therefore, based on practical application scenarios and user groups, it can provide targeted recognition results for the intelligent electric toothbrush, and adjust oral cleaning services by the intelligent electric toothbrush, further improving the applicability of the oral health system. In optional embodiments, the process for determining the position information of caries and/or dental calculus by the target detection algorithm in the recognition unit 201 includes the following steps:

segment the received oral image to obtain S×S blocks;

set multiple boxes in each block;

evaluate each box, including whether there is a target object in the box and the category of the target object when there is one in the box;

and delete boxes that do not have any target object and determine the positions of the boxes that have a target object;

In optional embodiments, the process for determining the severity grading information of caries and/or dental calculus through convolutional neural networks in the recognition unit 201 includes the following steps:

segment the oral image based on the position information of dental caries and/or dental calculus determined through target detection algorithms to obtain a tooth image with a target object which is dental caries and/or dental calculus;

use convolutional neural networks to grade the tooth image, different levels correspond to the severities of different target objects;

and output classification confidence, the higher the classification confidence, the higher the accuracy of the category evaluation of the corresponding target object.

The method for determining the position information of dental caries and/or dental calculus through object detection algorithms and the method for determining the severity grading information of dental caries and/or dental calculus

through convolutional neural networks in the above recognition unit 201 can be referred to in the instruction manual for details.

In optional embodiments, the server 2 further comprises a tooth information acquisition module and an oral mucosal information acquisition module, wherein the tooth information acquisition module is used to obtain the number and shape of the user's teeth based on oral images, and the oral mucosal information acquisition module is used to determine the presence of the user's oral mucosa based on oral cavity images. Therefore, the number of teeth, tooth morphology and oral mucosal condition of the user can serve as the basis for determining tooth cleaning parameters, in order to comprehensively grasp the user's oral health status and improve the user experience.

In optional embodiments, the tooth information acquisition module can determine the user's age group based on the number and morphology of teeth.

In optional embodiments, the parameter determination unit 108 set in the intelligent electric toothbrush 1 or server 2 can adjust the brushing duration in the teeth cleaning parameters based on the user's age group and number of teeth of children, the elderly and the user with fewer teeth, to protect the gum health in the edentulous area by reducing the brushing duration.

In optional embodiments, the server 2 further comprises an oral health report generation module for generating oral health reports based on the recognition result, and a second communication unit that can be used to send the oral health report to the designated terminal. Specifically, the designated terminal is the user associated mobile terminal or PC terminal.

In optional embodiments, the oral health report may include the user oral problem type, grading information, and related oral images. Therefore, based on user oral images and server analysis results, it can conduct long-term systematic tracking and analysis of user oral health status, obtain user oral problems and development trends, prevent oral diseases or enable the user to timely treat related oral diseases, and improve user experience.

In optional embodiments, the server 2 compares multiple oral health reports, generates oral health trend reports, and sends oral health trend reports to the designated terminal. Therefore, it provides the user with the comparative information on oral health status, allowing the user to have a more intuitive understanding of the oral health status.

It should be noted that the system provided by the above embodiment only provides examples of the division of each functional module when implementing its functions. In practical applications, it can allocate the above functions to different functional modules according to needs, that is, divide the internal structure of the device into different functional modules to complete all or part of the functions described above.

### Embodiment 2

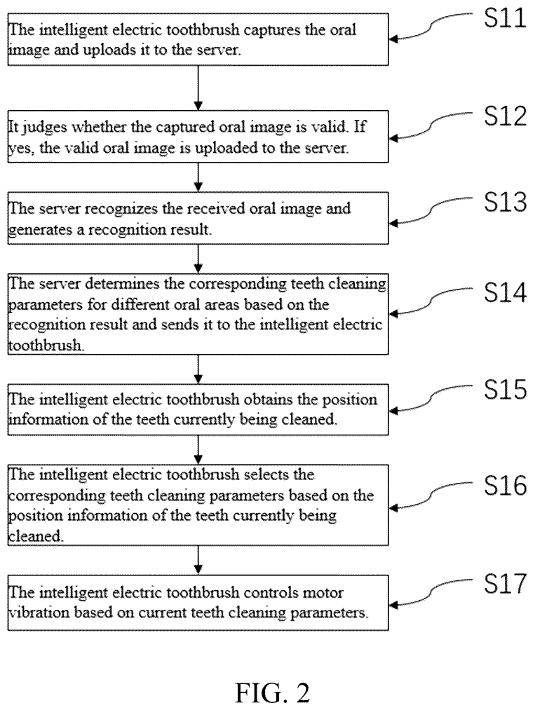

FIG. 2 shows the control method of the intelligent electric toothbrush based on the artificial intelligence image recognition to adjust the electric toothbrush provided in the embodiment of the application, which is applied to any oral health management system based on artificial intelligence image recognition to adjust the electric toothbrush in the above embodiment, including the following steps:

S11: the intelligent electric toothbrush captures oral images,

S12: the intelligent electric toothbrush judges whether the captured oral image is valid. If yes, the valid oral image is uploaded to the server. If not, the corresponding voice prompt is issued.

S13: the server recognizes the received oral image and generates the recognition result. The recognition result at least includes the information on the position of the teeth, information on the presence of caries and/or dental calculus, and information on the severity grading of caries and/or dental calculus.

S14: the server determines the corresponding teeth cleaning parameters for different oral areas based on the recognition result

and sends them to the intelligent electric toothbrush, or the server sends the recognition result to the intelligent electric toothbrush. The intelligent electric toothbrush determines tooth cleaning parameters corresponding to different oral areas, including brushing duration and/or vibration frequency, based on the recognition result.

S15: the intelligent electric toothbrush obtains the position information of the teeth currently being cleaned.

S16: the intelligent electric toothbrush selects the corresponding teeth cleaning parameters based on the position information of the teeth currently being cleaned.

cleaned.

S17: the intelligent electric toothbrush controls motor vibration based on current teeth cleaning parameters.

In optional embodiments, the intelligent electric toothbrush can communicate with the server through a wireless network or Bluetooth network.

In optional embodiments, in step S11, the intelligent electric toothbrush can capture oral images through a miniature wide-angle camera which is arranged on the intelligent electric toothbrush. The lens of the miniature wide-angle camera can be made of crystal glass.

In optional embodiments, when the intelligent electric toothbrush captures oral images in step

S11, it may include turning on the light replenishing module before acquisition.

In optional embodiments, in step S12, the intelligent electric toothbrush determines whether the oral image is valid, including determining whether the oral image is clear, determining whether the oral image is overexposed or overdark, and determining whether the imaging angle of the oral image is appropriate. Specifically, presetting the related parameter thresholds of the oral image, including clarity, exposure and imaging angle. Based on the preset parameter thresholds, the various conditions for the validity of the oral image are sequentially judged. If the oral image meets the requirements of clarity, moderate exposure and imaging angle, the oral image is judged to be valid and sent to the server for recognition. If the oral image does not meet at least one of the above three judgment conditions, it is determined that the oral image is invalid.

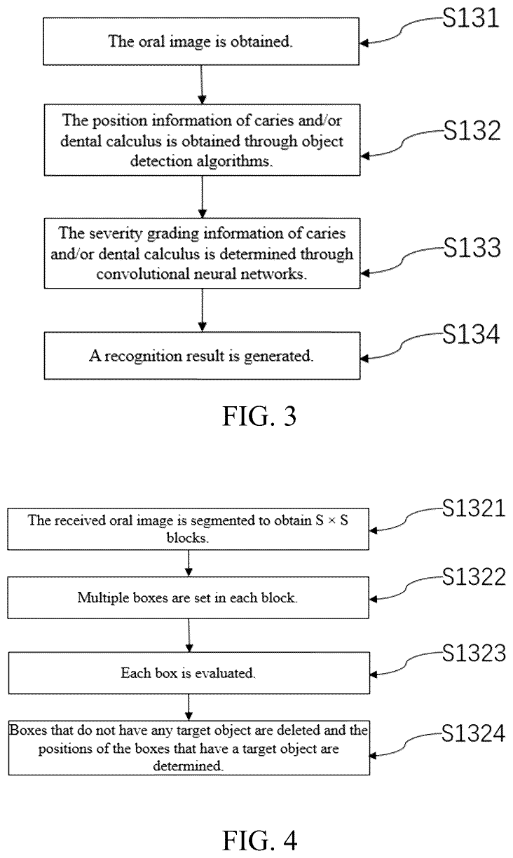

Referring to FIG. 3, step S13 can specifically include the following steps:

S131: obtain the oral image;

S132: determine the position information of caries and/or dental calculus through object detection algorithms;

S133: determine the severity grading information of caries and/or dental calculus through convolutional neural networks;

S134: and generate a recognition result.

In optional embodiments, referring to FIG. 4, step S132 specifically includes the following:

S1321: segment the received oral image to obtain S×S blocks;

S1322: set multiple boxes in each block;

S1323: evaluate each box, including whether there is a target object in the box and the category of the target object in the box;

Specifically, the category of the target object can be dental caries and/or dental calculus.

S1324: delete the box that does not have a target object and determine the position of the box that has a target object. The position of the box includes four values: the center point x value (bx) and y value (by), as well as the width (bw) and height (bh) of the box.

In optional embodiments, the object detection algorithm in step S132 can be the YOLOv5 algorithm.

Specifically, at the input end, YOLOv5 uses Mosaic data augmentation to concatenate some images together to generate new images, resulting in a larger number of images. In algorithm training, YOLOv5 can adaptively minimize the black edges after image scaling when inputting the training set images.

When determining the position of the box containing the target object, YOLOv5 predicts bx, by, bw and bh by predicting tx, ty, tw and th, the relationship is as follows:

bₓ=σ(tₓ)+Cₓ

b_{y}=σ(t_{y})+C_{y}

b_{w}=P_{w}e^{tw}

bₕ=Pₕe^{th}

Wherein, tx, ty, tw and th are predicted values, cx and cy are the coordinates of the upper left corner of the target object frame relative to the entire oral image, and pw and ph are the width and height of the target object frame.

At the output end, the GIOU-loss function is used to optimize the model parameters, the formula is as follows:

$${Loss}_{GIOU} = 1 - GIOU = 1 - ( IOU - \frac{\vert C ∖ ( AUB ) \vert}{\vert C \vert} )$$

Wherein, A and B are the boxes of the target object and the boxes of the real object, respectively, and IOU is the intersection and union of A and B, while C is the smallest bounding rectangle of A and B;

The overall loss (LOSS) function can be written as:

$${λ}_{coord} {\sum }_{i = 0}^{{S}^{2}} {\sum }_{j = 0}^{B} {1}_{ij}^{obj} [ {( {b}_{{x}_{i}} - )}^{2} + {( {b}_{{x}_{i}} - )}^{2} ] + {λ}_{coord} {\sum }_{i = 0}^{{S}^{2}} {\sum }_{j = 0}^{B} {1}_{ij}^{obj} [ {( \sqrt{{b}_{{w}_{i}}} - \sqrt{} )}^{2} + {( \sqrt{{b}_{{h}_{i}}} - \sqrt{} )}^{2} ] + {\sum }_{i = 0}^{{S}^{2}} {\sum }_{j = 0}^{B} {1}_{ij}^{obj} {( {C}_{i} - {\hat{C}}_{i} )}^{2} + {λ}_{noobj} {\sum }_{i = 0}^{{S}^{2}} {\sum }_{j = 0}^{B} {1}_{ij}^{noobj} {( {C}_{i} - {\hat{C}}_{i} )}^{2}$$

Wherein, bₓ, b_{y}, b_{w} and bₕ are predicted values, [symbol] , [symbol] , [symbol] , and [symbol] are labeled values, Cᵢ and Ĉᵢ are the confidence levels of predicted and labeled values, respectively. lᵢⱼ^{obj.} is a control function, indicating the presence of the object in the jth predicted box of grid i. lᵢⱼ^{noobj.} indicates that there is no object in the jth predicted box of grid i, λcoord and λ, Noobj are the two hyperparameters introduced to increase the weight of the object box containing the detection target.

In optional embodiments, due to the introduction of more boxes when using the YOLOv5 algorithm, non-maximum suppression (NMS) operation can be used to remove boxes of overlapping and repetitive target objects.

As a result, efficient and high-precision intelligent screening for dental caries and/or dental calculus is achieved.

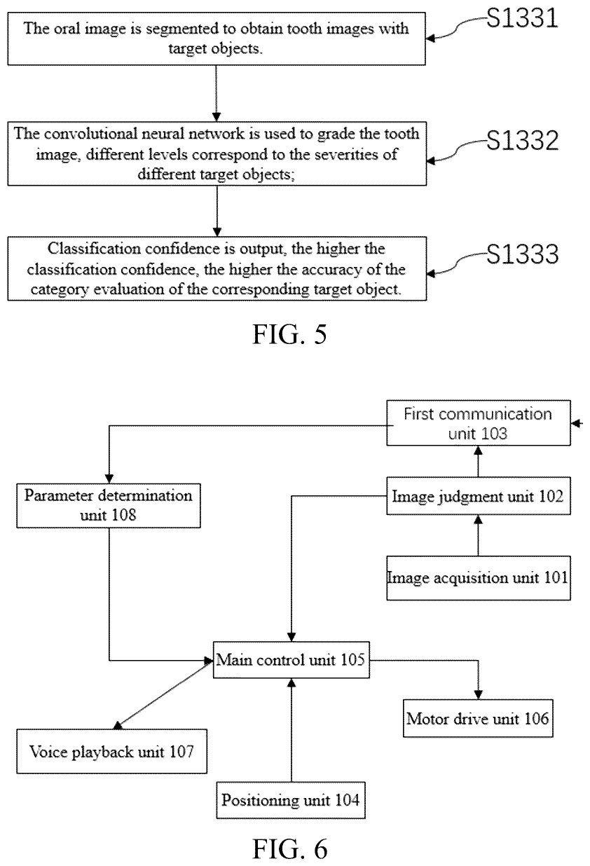

In optional embodiments, referring to FIG. 5, step S133 specifically includes the following:

S1331: segment the oral image based on the position information of dental caries and/or dental calculus determined through target detection algorithms to obtain a tooth image with a target object which is dental caries and/or dental calculus;

S1332: use convolutional neural networks to grade the tooth image, different levels correspond to the severities of different target objects;

In optional embodiments, the convolutional neural network is used to classify the severity of the target object,

which can be mild 0, moderate 1, and severe 2. From this, the severity of dental caries and/or dental calculus can be

determined.

S1333: output classification confidence. The higher the classification confidence, the higher the accuracy of the corresponding target object's category evaluation.

In optional embodiments, the convolutional neural network is used to output the classification confidence of each tooth image with a target object in step S1331. The higher the classification confidence level, the higher the accuracy of the classification evaluation result of the target object based on the convolutional neural network.

It can filter the recognition results based on the actual situation using classification confidence.

In optional embodiments, step S134 specifically includes generating the recognition result based on the output results of

steps S132 and S133.

In optional embodiments, in step S13, the recognition result may also include only the position information of the teeth, information on whether the teeth have caries, and severity grading information of the caries, or only the position information of the teeth, information on whether the teeth have dental calculus, and severity grading information of the dental calculus.

In optional embodiments, step S14 may further include: the server obtains the information on the number of user teeth, tooth morphology and the presence of the user's oral mucosa based on oral images.

In optional embodiments, step S14 may further include: the server determines the user age group based on the number and morphology of the teeth.

In optional embodiments, in step S14, when determining the teeth cleaning parameters, it can adjust the brushing duration in the teeth cleaning parameters for children, the elderly and users with fewer teeth based on the user's age group and number of teeth. By reducing the brushing duration, it can protect the gum health in the edentulous area.

In optional embodiments, in step S15, the intelligent electric toothbrush can obtain the position information of the teeth currently being cleaned through a gyroscope which is used to obtain the position, movement trajectory, and acceleration parameters of the intelligent electric toothbrush. The posture parameters during the use of the intelligent electric toothbrush can be used to determine the position information of the teeth being cleaned.

In optional embodiments, the method further includes the following: the server generates an oral health report based on the recognition result and sends the oral health report to a designated terminal. The oral health report can include user oral problem types, grading information and related oral images.

In optional embodiments, the method further includes the following: the server generates an oral health report based on the recognition result and sends the oral health report to a designated terminal. Oral health reports can include user oral problem types, grading information and related oral images.

In optional embodiments, the method further includes the following: the server compares multiple oral health reports, generates an oral health trend report, and sends the oral health trend report to a designated terminal.

In optional embodiments, the method further includes the following: the intelligent toothbrush takes photos and/or brushes teeth and/or plays voice based on the user's button operation.

The advantages of the disclosure are as follows: the accurate oral health information related to dental caries, dental calculus, etc. of the user is obtained by obtaining the user's oral image, and the recognition and analysis of the oral image is obtained through object detection algorithms and convolutional neural networks. The intelligent electric toothbrush is controlled accordingly, providing targeted oral cleaning services for the user, improving the cleaning effect of the intelligent electric toothbrush, and improving the user experience. The judgement of the intelligent electric toothbrush on the validity of the oral image avoids adverse effects on recognition results caused by unclear oral images, overexposure or darkness of images, and imaging angle issues, thereby further improving the accuracy of recognition results. Through the analysis of invalid images by the image judgment unit, the voice playback unit is controlled to play related voice data, providing prompts to the user in the form of voice, facilitating the user to obtain more intuitive guidance and obtaining effective oral images. On the one hand, it further improves the accuracy of the recognition result, and on the other hand, it improves the user experience.

In addition, the above method embodiments and system embodiments belong to a unified concept, and their specific processes and related advantages can be seen in the system embodiments, which are not repeated here.

### Embodiment 3

The intelligent electric toothbrush provided in an embodiment of the application, referring to FIG. 6, is applied to any of the above-mentioned oral health management systems based on artificial intelligence image recognition to adjust the electric toothbrush, which can specifically comprise the following:

An image acquisition unit 101 for capturing oral images;

an image judgment unit 102 for determining whether the captured oral image is a valid oral image. If yes, the image is sent by the first communication unit to the server for recognition. If not, the reason for the invalid image is sent to the main control unit.

a first communication unit 103 used to the send valid oral image to the server and receive recognition results or dental cleaning parameters sent back by the server. The recognition result at least includes the information on the position of the teeth, information on the presence of caries and/or dental calculus, and information on the severity grading of caries and/or dental calculus. The teeth cleaning parameters include brushing duration and/or vibration frequency;

a positioning unit 104 used to obtain the position information of the teeth being cleaned and send the information to the main control unit;

a main control unit 105 used to receive the reason for the invalid image, select the corresponding voice data in the voice database based on the reason for the invalid image, and select the corresponding teeth cleaning parameter based on the position information of the teeth being cleaned and convert it into a control signal and send it to the motor drive unit in real time;

a motor drive unit 106 used to connect the motor and drive the motor to vibrate based on the control signal of the main control unit;

and a voice playback unit 107 used for voice playback based on the voice data selected by the main control unit.

The optional embodiment also comprises the following:

A button operation module for receiving the user's button operation signal and sending it to the main control unit. In addition, the main control unit can receive the button operation signal and convert it into a corresponding control signal to be sent to the image acquisition unit, motor drive unit and voice playback unit.

a touch screen unit 207 used to operate various parameter options of the intelligent toothbrush, while presenting the incentive system information for brushing feedback.

In addition, the intelligent electric toothbrush provided in this embodiment belongs to the same concept as the system embodiment. The specific implementation process and related advantages can be seen in the system embodiment, which are not repeated here.

The advantages of the disclosure are as follows: the intelligent electric toothbrush obtains a user's oral image and receives the recognition results or tooth cleaning parameters sent back by the server. The intelligent electric toothbrush is controlled accordingly, providing targeted oral cleaning services for the user, improving the cleaning effect of the intelligent electric toothbrush, and improving the user experience. The judgement of the intelligent electric toothbrush on the validity of the oral image avoids adverse effects on recognition results caused by unclear oral images, overexposure or darkness of images, and imaging angle issues, thereby further improving the accuracy of recognition results. Through the analysis of invalid images by the image judgment unit, the voice playback unit is controlled to play related voice data, providing prompts to the user in the form of voice, facilitating the user to obtain more intuitive guidance and obtaining effective oral images. On the one hand, it further improves the accuracy of the recognition result, and on the other hand, it improves the user experience.

Each embodiment in this specification is described in a progressive manner, and the same and similar parts between each embodiment can be referred to each other. Each embodiment focuses on the differences from other embodiments. Especially for embodiments of devices, equipment and storage media, as they are basically similar to method embodiments, the description is relatively simple. Please refer to the partial explanation of method embodiments for related details.

Ordinary technical personnel in this field can understand that all or part of the steps to implement the above embodiments can be completed through hardware, or can be instructed to be completed by related hardware through programs. The programs can be stored in a computer-readable storage medium, which can be read-only memory, magnetic disk or optical disk, etc.

The above are only preferred embodiments of the disclosure and are not intended to limit it. Any modifications, equivalent substitutions, improvements, etc. made within the spirit and principles of the disclosure shall be included in the scope of protection of the disclosure.

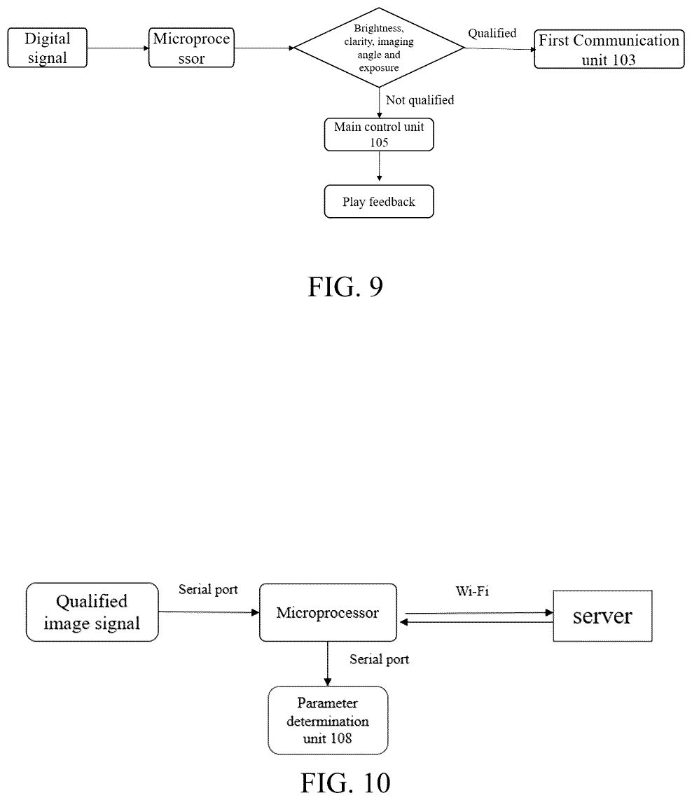

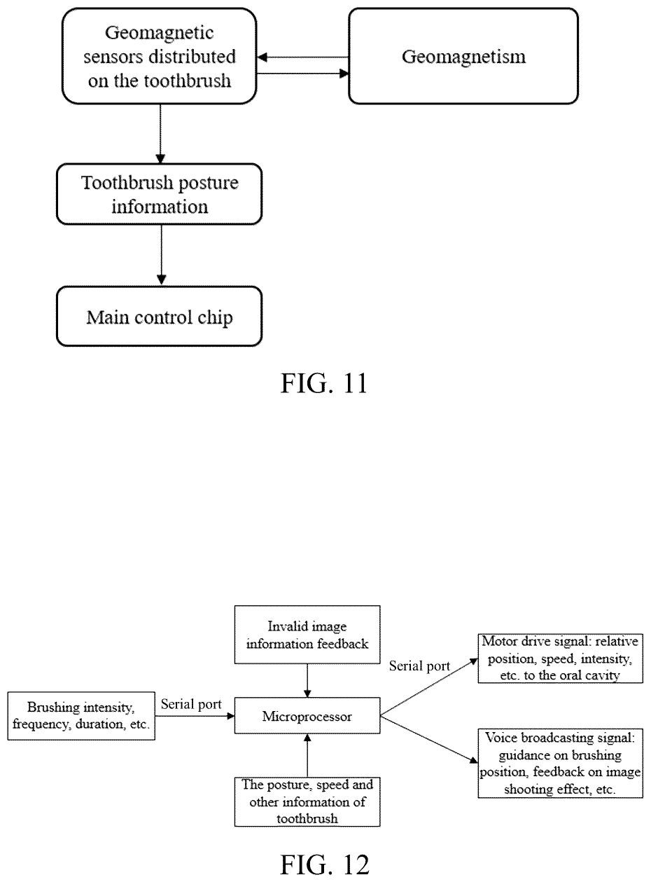

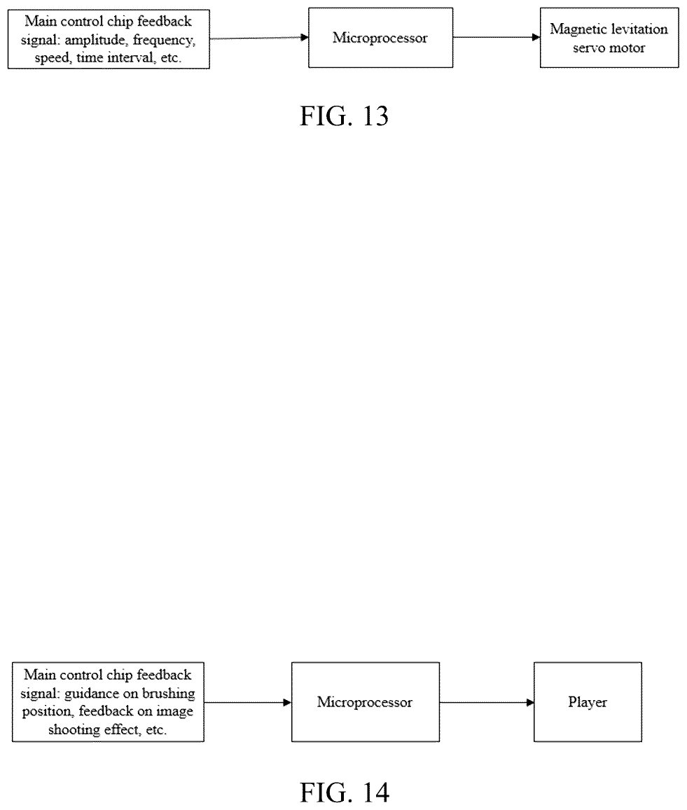

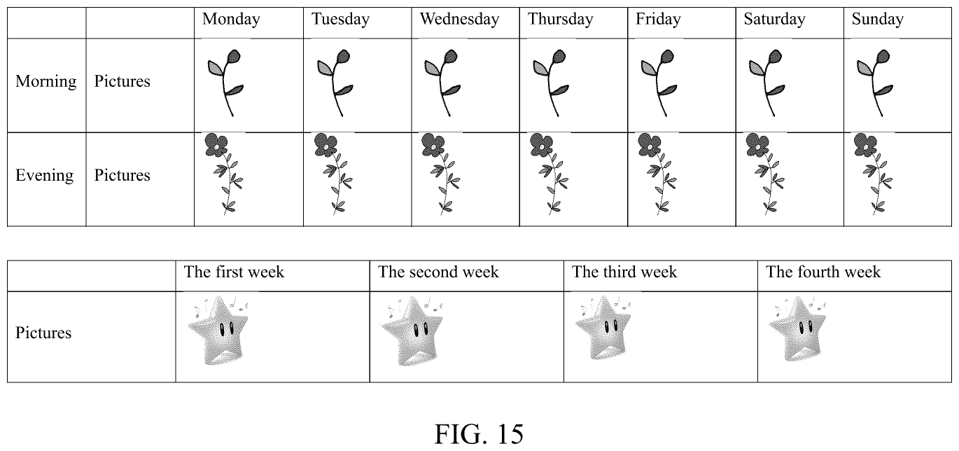

1. An oral health management system comprising an intelligent electric toothbrush and a server;
wherein,
the intelligent electric toothbrush comprises the following:
a) an image acquisition unit (101) for capturing oral images, with the specific technical details including: an optical lens is used to capture oral image information; if necessary, light replenishing is provided; then the optical information is converted into electrical and digital signals through a photoelectric sensor, as shown in FIG. 8;
b) an image judgment unit (102), used to determine whether the captured oral image is a valid one; if yes, the image is sent by the first communication unit to the server for recognition; if not, the reason for the invalid image is sent to the main control unit; the process of determining whether the captured oral image is a valid one is as follows: preset parameter thresholds for the clarity, exposure and imaging angle of the oral image; according to the preset parameter thresholds, judge whether the clarity, exposure and imaging angle of the oral image are qualified; if the clarity, exposure and imaging angles of the oral image are all qualified, the oral image is judged to be valid, otherwise the oral image is judged to be invalid; the standards for judging whether the imaging angle of the oral image is qualified is whether the imaging angle of the oral image is within the range of greater than 50° and less than or equal to 90 ( ) if yes, the imaging angle of the oral image is qualified; if not, the imaging angle of the oral image is unqualified; the working principle and process of the image judgment unit are shown in FIG. 9;
c) a first communication unit 103 used to send the valid oral image to the server and receive recognition results or dental cleaning parameters sent back by the server. The recognition result at least includes the information on the position of the teeth, information on the presence of caries and/or dental calculus, and information on the severity grading of caries and/or dental calculus. The teeth cleaning parameters include brushing duration and/or vibration frequency. The working principle and flowchart of the first communication unit are shown in FIG. 10.
d) a positioning unit (104) used to obtain the position information of the teeth being cleaned and send the information to the main control unit. The working principle and flowchart of the positioning unit are shown in FIG. 11;
e) a main control unit (105) used to select corresponding teeth cleaning parameters based on the position information of the teeth being cleaned and convert them into control signals to be sent to the motor drive unit in real time. In addition, upon receiving the reason for the invalid image sent by the image judgment unit, the main control unit (105) selects the corresponding voice data in the voice database based on the reason for the invalid image. The working principle of the main control unit is shown in FIG. 12;
f) a motor drive unit (106) used to connect the motor and drive the motor to vibrate based on the control signal of the main control unit. The working principle and flowchart of the motor drive unit are shown in FIG. 13;
g) a voice playback unit (107) used for voice playback based on the voice data selected by the main control unit, as shown in FIG. 14;
h) and an intelligent interactive unit (109) used to provide timely feedback on the user' brushing information. Brushing teeth in the morning displays one small red flower, while brushing teeth in the evening rewards one small red flower. Brushing teeth continuously for a week can be exchanged for a small star. In this way, the user is motivated to develop good habits, as shown in FIG. 15.

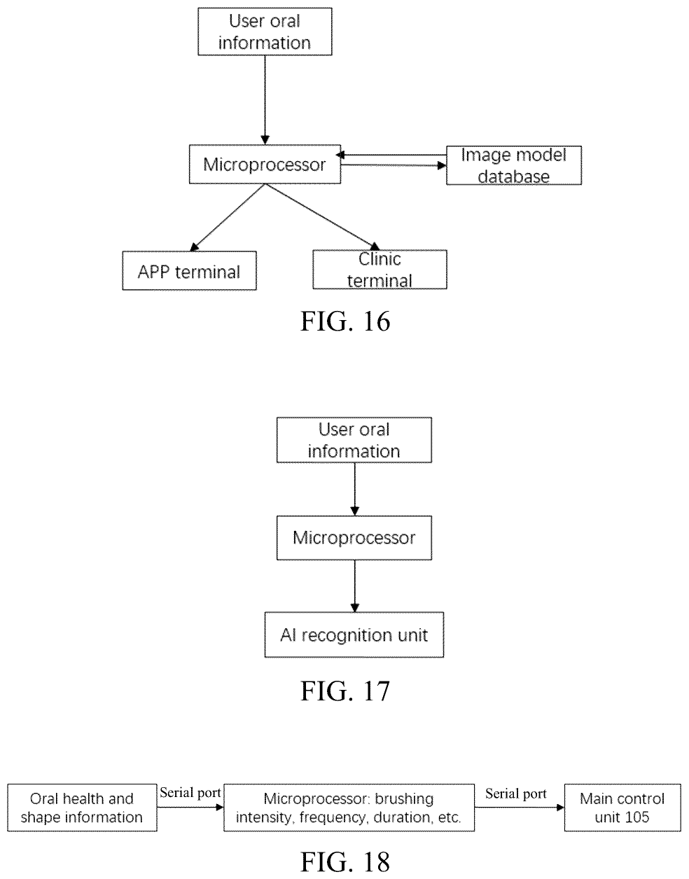

2. The oral health management system of claim 1, wherein the server comprises
i. a recognition unit (201) for recognizing the received oral image and generating a recognition result. The recognition method includes the following steps: obtaining an oral image, determining the position information of caries and/or dental calculus through object detection algorithms, determining the severity grading information of caries and/or dental calculus through convolutional neural networks, and generating a recognition result, as shown in FIG. 16;
ii. a second communication unit (202) for receiving the oral image sent by the intelligent electric toothbrush and sending a recognition result or tooth cleaning parameter to the intelligent electric toothbrush, as shown in FIG. 17;
iii. and a parameter determination unit (108) for determining tooth cleaning parameters corresponding to different oral areas based on the recognition result. The parameter determination unit is set on the intelligent electric toothbrush or server. In the recognition unit of the server, the process of determining the position information of dental caries and/or dental calculus through the object detection algorithms includes the following steps: segment the received oral image to obtain S×S blocks; set multiple boxes in each block; evaluate each box, including whether there is a target object in the box and the category of the target object when there is one in the box; delete boxes that do not have any target object and determine the positions of the boxes that have a target object; and in the recognition unit of the server, the severity grading information of dental caries and/or dental calculus is determined through convolutional neural networks, which includes segment the oral image based on the position information of dental caries and/or dental calculus determined through target detection algorithms to obtain a tooth image with a target object which is dental caries and/or dental calculus; use convolutional neural networks to grade the tooth image, different levels correspond to the severities of different target objects; and output classification confidence, the higher the classification confidence, the higher the accuracy of the category evaluation of the corresponding target object, as shown in FIG. 18.

3. The oral health management system based on artificial intelligence image recognition to adjust the electric toothbrush according to claim 1 is characterized in that the intelligent electric toothbrush further comprises a button operation unit for receiving the user's button operation signal and sending it to the main control unit; and a main control unit that can receive the button operation signal and convert it into a corresponding control signal to be sent to the image acquisition unit, motor drive unit and voice playback unit.

4. The oral health management system based on artificial intelligence image recognition to adjust the electric toothbrush according to claim 1, characterized in that the server further comprises an oral health report generation module for generating oral health reports based on the recognition result, and a second communication unit that can be used to send the oral health report to the designated terminal.

5. A control method based on the artificial intelligence image recognition to adjust the intelligent electric toothbrush, characterized in that the oral health management system based on the artificial intelligence image recognition to adjust the intelligent electric toothbrush according to claim 1 includes the following steps: the intelligent electric toothbrush captures oral images, and judges whether the captured oral image is valid; if yes, the valid oral image is uploaded to the server; if not, the corresponding voice prompt is issued; the steps for judging whether the captured oral image is valid include: presetting parameter thresholds for oral image clarity, exposure and imaging angle; according to the preset parameter thresholds, whether the clarity, exposure and imaging angle of the oral image are qualified is judged; if the clarity, exposure and imaging angle of the oral image are qualified, it is judged that the oral image is valid; otherwise, it is judged that the oral image is invalid; the server recognizes the received oral image and generates the recognition result; the recognition result at least includes the position information of the teeth, information on the presence of caries and/or dental calculus, and information on the severity grading of caries and/or dental calculus; the server determines the corresponding teeth cleaning parameters for different oral areas based on the recognition result and sends them to the intelligent electric toothbrush, or the server sends the recognition result to the intelligent electric toothbrush; the intelligent electric toothbrush determines tooth cleaning parameters corresponding to different oral areas, including brushing duration and/or vibration frequency, based on the recognition result; the intelligent electric toothbrush obtains the position information of the teeth currently being cleaned, and selects the corresponding teeth cleaning parameters based on the position information of the teeth currently being cleaned; the intelligent electric toothbrush controls motor vibration based on current teeth cleaning parameters; wherein, the server recognizes the received oral image and generates the recognition result; the recognition method includes the following steps: obtaining an oral image, determining the position information of caries and/or dental calculus through object detection algorithms, determining the severity grading information of caries and/or dental calculus through convolutional neural networks, and generating the recognition result; the process of determining the position information of dental caries and/or dental calculus through the object detection algorithms includes the following steps: segment the received oral image to obtain S×S blocks; set multiple boxes in each block; evaluate each box, including whether there is a target object in the box and the category of the target object when there is one in the box; delete boxes that do not have any target object and determine the positions of the boxes that have a target object, and the severity grading information of dental caries and/or dental calculus is determined through convolutional neural networks includes the following steps: segment the oral image based on the position information of dental caries and/or dental calculus determined through target detection algorithms to obtain a tooth image with a target object which is dental caries and/or dental calculus; use convolutional neural networks to grade the tooth image, different levels correspond to the severities of different target objects; and output classification confidence, the higher the classification confidence, the higher the accuracy of the category evaluation of the corresponding target object.

6. The control method based on the artificial intelligence image recognition to adjust the intelligent electric toothbrush according to claim 4, characterized in that the intelligent electric toothbrush performs one or more of the photo taking, brushing, and voice playback operations according to the user's button operation.

7. The control method based on the artificial intelligence image recognition to adjust the intelligent electric toothbrush according to claim 4, characterized by including the following steps: the server generates an oral health report based on the recognition result; and the server sends the oral health report to the designated terminal.

8. An intelligent electric toothbrush applied to the oral health management system based on artificial intelligence image recognition to adjust the electric toothbrush according to claim 1, characterized by comprising the following: an image acquisition unit for capturing oral images; an image judgment unit for determining whether the captured oral image is a valid oral image; if yes, the image is sent by the first communication unit to the server for recognition; if not, the reason for the invalid image is sent to the main control unit; the process of determining whether the captured oral image is a valid oral image is as follows: preset parameter thresholds for the clarity, exposure and imaging angle of the oral image; according to the preset parameter thresholds, the clarity, exposure and imaging angle of the oral image are judged to be qualified; if the clarity, exposure and imaging angles of the oral image are all qualified, the oral image is judged to be valid, otherwise the oral image is judged to be invalid; a first communication unit used to the send the valid oral image to the server and receive the recognition result or dental cleaning parameters sent back by the server; the recognition result at least includes the information on the position of the teeth, information on the presence of caries and/or dental calculus, and information on the severity grading of caries and/or dental calculus; the teeth cleaning parameters include brushing duration and/or vibration frequency; a positioning unit used to obtain the position information of the teeth being cleaned and send the information to the main control unit; a main control unit used to receive the reason for the invalid image, select the corresponding voice data in the voice database based on the reason for the invalid image, and select the corresponding teeth cleaning parameters based on the position information of the teeth being cleaned, convert them into control signals, and send them to the motor drive unit in real-time; a motor drive unit used to connect the motor and drive the motor to vibrate based on the control signal of the main control unit, and a voice playback unit used for voice playback based on the voice data selected by the main control unit.

9. The intelligent electric toothbrush according to claim 7 is characterized by further comprising a button operation module for receiving the user's button operation signal and sending it to the main control unit; and a main control unit that can receive the button operation signal and convert it into a corresponding control signal to be sent to the image acquisition unit, motor drive unit and voice playback unit.

10. The intelligent electric toothbrush according to claim 9, further characterized by a touch screen system for interactive stimulation; if the consumer brushes teeth once a day, the consumer will be rewarded by a small red flower displayed on the screen each day, and the accumulated small red flowers for a week is exchanged for a small star, making children gain interest of brushing teeth.
